(19) United States
(12) Patent Application Publication
(10) Pub. No.: US 2013/0023787 A1
(43) Pub. Date: Jan. 24, 2013

(54) Hearing screener method and device with online scheduling and physical referral

(71) Applicant: Kathryn R. Dowd, Charlotte, NC (US)

(72) Inventor: Kathryn R. Dowd, Charlotte, NC (US)

(21) Appl. No.: 13/373,498

(22) Filed: Nov. 16, 2011

Related U.S. Application Data
(60) Provisional application No. 61/572,782, filed on Jul. 21, 2011.

(51) Int. Cl. A61B 5/12; A61B 5/11; A61B 3/032

(52) U.S. Cl. 600/558; 600/559; 600/595

(57) ABSTRACT

An automated screener device screens hearing, schedules an appointment, and produces a physician order upon a patient failure of the screening. The screener includes a computer with a touch screen to facilitate responses to a short questionnaire and to auditory stimuli. A set of noise cancelling head phones are used by the patient during the hearing screening. The computer has an associated printer which prints test results. A failed screening test causes production of a printed report having signature line for physician referral. An appointment schedule appears on the touch screen immediately allowing the patient to schedule an appointment with an area audiologist. The method of operation is also disclosed.

### CROSS REFERENCE TO RELATED APPLICATION

This application claims the benefit of U.S. Provisional Application No. 61/572,782, filed Jul. 21, 2011.

### FIELD OF THE INVENTION

The present invention relates to a method and apparatus for screening a person to determine whether hearing problems exist on an individual basis. The apparatus is a hearing screener desktop device or kiosk having online web based scheduling and report printing capabilities

### BACKGROUND OF THE INVENTION

Presently available hearing screening devices are usually located in audiologist or medical offices, or in clinics. Such screening devices are not readily available to the public. Those that are available do not print out results with a doctor's signature line as needed at this point for insurance (Medicare, Medicaid, or commercial insurance) coverage of future diagnostic hearing testing. They are not linked online to a specific nearby audiology or hearing clinic schedule where an appointment can be made immediately upon an indication of failure of the hearing screening. Current screening devices do not utilize a touch screen for response to hearing screening stimuli. Present practice does not incorporate a web portal with an internet based scheduler for a local contracted clinic where, immediately on failing the hearing screening, a patient may schedule a convenient, time to have a full hearing evaluation. Present practice does not provide printed reports, online scheduling and automated testing in one apparatus for screening hearing.

### SUMMARY OF THE INVENTION

The invention provides a hearing screener device with capacity to screen hearing, schedule an appointment, and produce a physician order upon failure of the screening. This composite screener can be used online or in physician and other health care provider offices, or it can be a kiosk-type device for use in a common area such as a mall, hospital, clinic, or pharmacy. The invention includes a computer with a touch screen to facilitate responses to a short questionnaire and to auditory stimuli. A set of noise cancelling head phones are connected (wired or wireless) to the computer to perform the hearing screening. The computer is attached (wired or wireless) to a printer which prints test results. Failed screening test results will produce a printed report having signature line for physician referral. The printout can be carried, faxed, or emailed to a physician for signature. An appointment schedule appearing on the screen immediately allows the patient to schedule an appointment with an area audiologist.

The present invention, referred to as AuDChek, allows both objective (by an automated test device) and subjective (by administering a questionnaire) screening of a person's hearing. An automated hearing test is self administered in about 10 minutes. A touch screen display is viewed by the person to be tested and receives responses to the test by the person touching appropriate portions of the screen. The software also provides a pop-up screen containing a contracted online clinic's appointment schedule, which pops up automatically upon failure of a screening test. This allows a diagnostic hearing evaluation to be scheduled to determine cause and remedial potential of the hearing problem. Screening results and appointment date and time are printed out for the chart of the person (hereinafter “patient”), and for medical approval for further testing for a patient who fails the screening. The audiology or hearing clinic is notified by email of the new appointment so a welcome letter and other forms can be mailed to the patient.

The invention will make unknown hearing problems known, and quickly route the patient to a qualified clinic to address the problems. Normally it takes a patient from 5 to 7 years to admit a hearing problem to oneself, and to become concerned enough about a hearing problem to act and get tested. The shortened time from screening to follow-up reduces the time that the hearing problem will remain an unknown problem affecting the patient's communication adversely on the job, within the family, or with a spouse. Improving access to hearing healthcare can reduce the depression, isolation and confusion that a patient experiences with an untreated hearing problem. The screening results are immediately available, with an immediate print out, and with immediate scheduling of an appointment for full evaluation, if needed. The value to the patient is quick assessment, service and treatment of the hearing problem.

The benefit to the contracting clinic is direct patient scheduling of qualified patients, i.e., those that failed the hearing screen. The clinic saves time and money over that presently required to find and schedule patients needing hearing healthcare services. More resources can then be spent on service needs such as diagnostic tests, hearing aids when appropriate, personal counseling, and aural rehabilitation.

### OBJECTS OF THE INVENTION

The principal object of the present invention is to provide an improved method of determining the hearing capability of a person.

Another object of the invention is to provide and conduct an automated hearing test and determine the responses to the test.

Another object of the invention is to provide an apparatus for screening the hearing capability of a person.

A further object of this invention is to provide a device that incorporates a touch screen for response to hearing screening stimuli.

Another object of the invention is to provide a device that prints out results with a doctor's signature line, as needed at this point for insurance coverage of future diagnostic hearing testing.

Another object of the invention is to provide a device that is linked online to a specific nearby clinic schedule where appointments can be made immediately upon failing a hearing screening.

Another object of the invention is to provide a device that incorporates a web portal with an internet based scheduler for a local contracted audiology or hearing clinic where, immediately on failing a hearing screening, a patient schedules a convenient, open time to have a full hearing evaluation.

Another object of the invention is to provide a device that provides printed reports, online scheduling and automated testing in a single apparatus for screening hearing.

### BRIEF DESCRIPTION OF THE DRAWING

The foregoing and other objects will become more readily apparent by referring to the following detailed description and the appended drawing in which:

FIG. 1 is a schematic drawing showing the relationship between local sites, audiology clinic, and the main server.

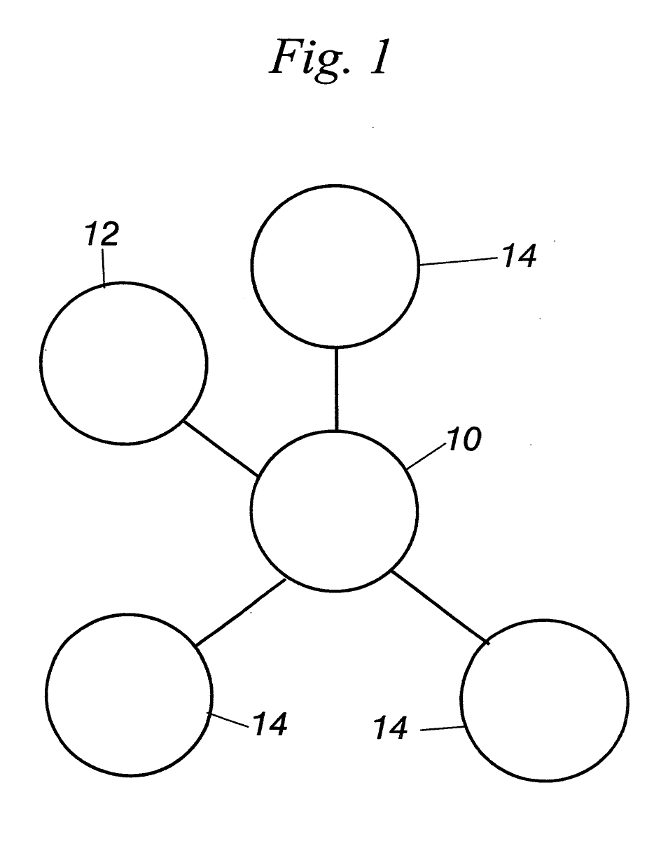

FIG. 2 is a schematic diagram showing the components of the invented hearing screener device.

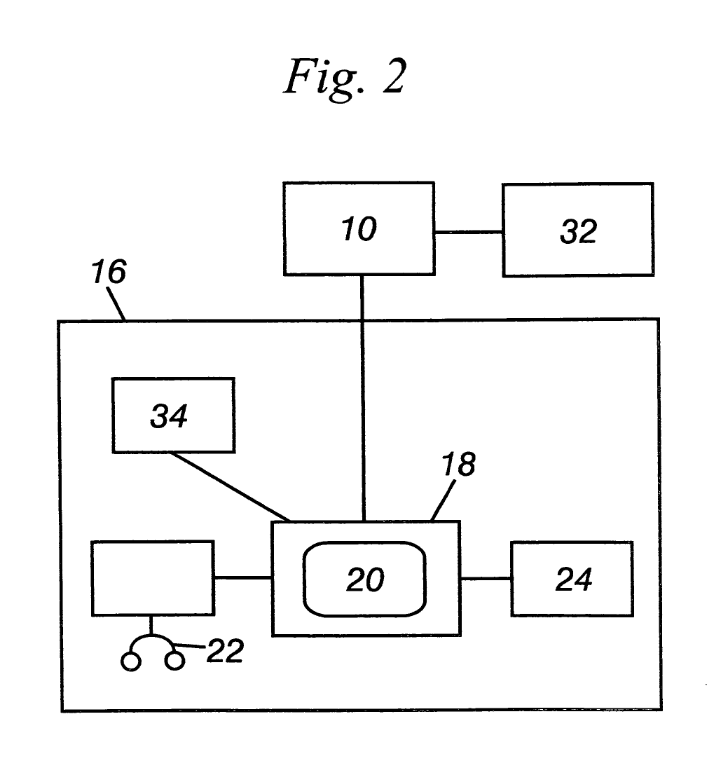

FIG. 3 is a schematic diagram showing the components of the invention utilized as a balance screener device.

FIG. 4 is a schematic diagram showing the components of the invention utilized as a vision screener device.

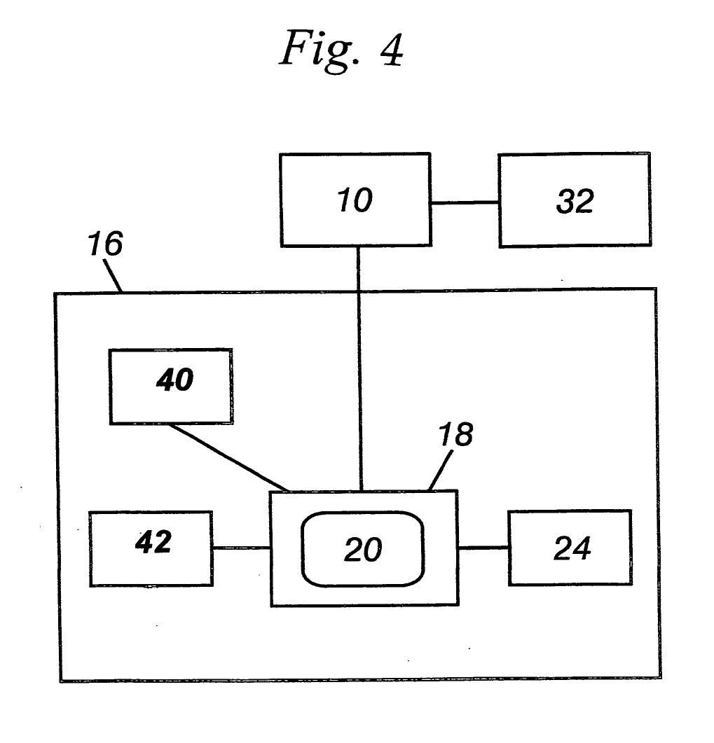

### DETAILED DESCRIPTION

The invention solves the problem of getting a patient screened for hearing problems, referred to a proper practitioner, and scheduled for an appointment with the practitioner, all in one sitting. Previous screening devices have not automatically scheduled an appointment for a diagnostic test (leaving the patient and doctor without knowledge of the nature and degree of hearing loss) nor have previous screeners produced the required physician order to ensure insurance reimbursement of the scheduled diagnostic evaluation.

A hearing screening kiosk can be a stand-alone instrument or apparatus, or an online portal. Consumers of the invention will be audiology/hearing offices which will be linked for scheduling purposes to the stand-alone kiosk or screening apparatus.

Referring now to the drawings, a main server (computer) 10 hosts a database and is linked to a scheduler in an affiliated office or clinic 12, such as an audiology office or other health care provider. The main server is connected to one or more local screening sites 14, which may be a kiosk or housing 16 having an automated hearing screener, including a central processing unit or computer, touch screen for patient responses, and interne access and connection.

The invented apparatus includes a computer 18 having a touch screen monitor 20, accompanying noise canceling headset with headphones 22, and associated printer 24. Software utilized in the invention includes a questionnaire with Y, N, S responses, a pure tone or NBN stimuli in Bekesy presentation. An automated hearing test protocol uses pure tone testing and the Hearing Handicap Inventory or similar scaled questionnaire.

All kiosk screeners are linked to a main server or CPU 10 that contains operational software. The server has the capacity to link to individual audiology office schedules and databases to manage patient data, appointments, and follow-up care.

The apparatus is a hearing test device mounted on a desktop computer or in a kiosk with touch screen. Potential locations for the device are in venues where medical, healthcare related or pharmacy services are performed.

The basic components of the apparatus of the invention are:

a computer device 18 with touch screen 20, noise cancelling headphones 22, and associated printer 24;

a secure internet web site 32 for housing the screening results and communicating with a contracted clinic's scheduling program; and

an automated hearing test protocol 34 using pure tone testing and/or the Hearing Handicap Inventory or a similar scaled questionnaire.

The AuDChek screening device is basically a laptop computer or a central processing unit (CPU) having a touch screen monitor, which can be incorporated into a kiosk 16 or it can be simply a computer presentation. A database section stored on a main server is completed with identifying information about the patient and medical history (DOB, Medical Doctor, chart numbers, associated diagnoses, etc.) An automated hearing screening protocol or program 34 is downloaded into the computer 18, along with the Hearing Handicap Inventory, a questionnaire of about 10 inquiries into the patient's hearing status which is administered under noise cancelling headphones. The automated hearing screening program is an objective assessment of hearing air conduction levels, while the questionnaire is a subjective assessment of the patient's insight into any hearing difficulties.

The actual screener/test device incorporates a touch screen computer (such as IPad or desktop) with blue tooth, and wired noise cancelling circumaural headphones. The screener is linked to an online web-based portal or database management site into which patient identifying information and related diagnosis information is entered. Such entry can be made by the patient or a healthcare worker on site.

The two parts of the hearing screening process are:

1) The patient answers a questionnaire about hearing health, the responses to which are recorded in the computer.

2) The patient responds to hearing test stimuli such as specific frequency tones or narrow band stimuli in an automated test mode, by touching a touch screen display, or by using a curser indicated on the screen, or by pressing a button which is connected with the computer to record the response. Alternatively, the patient may voice a response, or use an Iphone or other interactive device connected to the computer. A suitable automated test is a Bekesy automated process.

The hearing health questionnaire may be any one of the following, or it may be another questionnaire available to the site operator.

TELEGRAM, developed by Linda M. Thibodeau, Ph.D, of the Callier Center at the University of Texas at Dallas, has a rating scale from 1 (no difficulty) to 5 (great difficulty) for situations such as Telephone, Employment, Legislation/employment, Entertainment, Groups, Recreation, Alarms, Members in household. The scale prioritizes 3 main problems to address from the 8 listening situations.

COSI: Client Oriented Scale of Improvement, developed by H. Dillon, A. James, and J. Ginis, of the National Acoustic Laboratories, Chatswood, Australia, ranks hearing needs in order of importance. The form is used again post hearing aid fitting to determine level of improvement.

HHIES: Hearing Handicap Inventory in the Elderly, developed by EAR Audiology, Inc., of San Diego, Calif., has 10 or more questions for the patient to rate the patient's sense of a hearing problem in a variety of situation.

COAT: Characteristics of Amplification Tool is a widely used form usually containing 9 questions which rank communication needs, personal preferences and expectations for hearing aids.

One or more of the following tests/questionnaires may be used to assess hearing:

1) An automated air conduction pure tone hearing test which provides a systematic presentation of tones, with responses by the subject regulating subsequent tone presentations. The test is shortened for hearing screening purposes.

2) A narrow band hearing test in which test stimuli are presented in a narrow band width centered around a pure tone, with narrow band stimuli located in the speech range.

3) A speech in noise test, such as the Quick Sin test, which presents sentences in speech-to-noise ratios of varying difficulties.

4) A speech word test in which words are presented in constant or varying intensity levels, and are used to assess speech threshold levels or word discrimination/understanding abilities.

5) Any other hearing assessment test which is available to audiologists.

To each question in the hearing assessment test, the patient responds to the test stimuli by touching the touch screen. Alternatively, the patient can respond by pressing a response button, or a key on a keyboard, by voicing a response when a test monitor is present or to voice recognition technology in the computer, by using a curser to indicate an answer on the response screen of the computer, or by utilizing an I-phone, or other interactive device linked to the computer.

The computer 18 determines, records, and indicates the results of the test as pass or fail. Each test which can be used in the hearing screening process contains pass/fail criteria within the test module. In air conduction and narrow band hearing tests, a ‘pass’, indicating that the hearing of the person tested is satisfactory, is generated and recorded when responses to tones/noise/speech are in the 0-25 db levels for the 4 central speech frequencies (0.5, 1, 2, 4 KHz). A ‘fail’ of the hearing screening test is generated and recorded when responses for any of the tones/noise/speech are to tones greater than 25 db. Results are shown in graphic representation of responses. Speech word tests and speech in noise tests incorporate a pass/fail criteria based on a predetermined percentage of words with a correct response. Noise level in the test location is taken into consideration. The noise level at the screening location is coded by the responder or healthcare worker as: 1) no noise, 2) slight noise, 3) moderate noise, 4) excessive noise. Moderate and excessive noise will invalidate the pass/fail criteria and test results.

Personal patient information is entered into the computer by the patient or an attendant, and it is encrypted and HIPAA (Health Insurance Portability and Accountability Act of 1996) compliant. Data collected includes patient's name, date of birth, address, phone, e-mail address, the name of the patient's personal physician. Upon completion of entry of the personal patient information, a checklist appears on the screen for the patient to indicate other significant medical diagnoses (vascular disease, hypothyroidism, chronic renal disease, cancer, ototoxic medications, etc., and preference for scheduling (am, pm, etc.)

The test results and patient information are securely transmitted from the test site to the audiology clinic, medical office or other healthcare office by internet, e-mail, fax or phone.

An important aspect of the invention is that the device is linked to an online scheduling portal. A patient who fails the screening test has an immediate opportunity to schedule an appointment with a local clinic in a web based scheduling portal. A program storing the patient information and test results in the central processing unit or server 10 is linked to a contracted audiology or medical clinic 12 that will provide further audiology testing, medical treatment and/or rehabilitation of the hearing loss.

After the screening, the device prints a report signifying pass or fail. A copy of the report is directed internally for the referring clinic patient records. The printed failure report has a signature line for the doctor's signature of approval, if necessary for a subsequent complete diagnostic hearing evaluation or other treatment. This printed report may be taken to a referral coordinator at check out to assist scheduling an appointment for the diagnostic evaluation with the audiology clinic to ensure that the patient is successful in setting an appointment if the online scheduling was not completed.

The patient obtains a signature from a doctor, or the form is sent to a doctor by electronic means for signature. The signed form is delivered to the audiology/hearing clinic 12. Alternatively, the signed form can be faxed from the local screening site 14 to the contracted clinic.

The software in the AuDChek device receives and records individual patient information in computer 18, which is transmitted to and maintained on server 10 linked to the contracted audiology clinic 12. The local site device or kiosk 14 includes a web based scheduling site 32: i.e., a portal for managing appointments. The online scheduling site can be the clinic's alternate reserved schedule. This will allow for clinic reserved times to be used only by AuDChek. The clinic receives notification of the private patient information at the time of scheduling.

The online scheduling portal generates appointments over the internet in real time for persons failing the hearing screen test. The screening kiosk 14 is linked by internet to a contracted local audiology clinic 12. A failed screening result causes a display to appear on the touch screen 20, which is the online screen of the clinic. The patient then selects a convenient appointment time by touching the appropriate portions of the touch screen. The clinic receiving the appointment has customized hours to receive appointments for diagnostic hearing evaluations.

The online portal to the schedule is managed directly onto the database management/scheduling system employed by the audiology clinic (e.g, Sycle or Tims), or to a stand-alone online schedule. The online portal is posted on the audiology clinic's website. Upon successful scheduling of an appointment, the computer generates an email confirmation to the patient, as well as an email notification to the clinic.

The scheduling portal is incorporated into the computer, which has a “button” or “box” on the touch screen 20 display that appears for selecting and booking an appointment, the computer being linked through the internet to the contracted audiology clinic. Such linking is accomplished by a secure internet connection according to industry best practices (NIST SP 800-53) and HIPAA regulations.

This invented apparatus will securely transmit and store Protected Health Information (“PHI”) between healthcare providers. In order to comply with Federal regulations including HIPAA and the HITECH Act, all applicable security standards are followed, including those based on NIST Special Publication 800-53, “Recommended Security Controls for Federal Information Systems and Organizations.” Risk management includes a documented development approach, operational plans development, and evaluation of threats, vulnerabilities, and impacts in order to identify important trends and decisions on where effort should be applied to eliminate or reduce threat capabilities and eliminate or reduce vulnerabilities. Such management of risk facilitates a consistent, comparable, and repeatable approach for security in the information system.

Information security requirements for the system will be satisfied with consideration of the risk management strategy of the organization, in light of the potential cost, schedule, and performance issues associated with the system acquisition, deployment, and operation.

### Alternative Embodiments

A similar kiosk can be utilized for balance screening, and for vision screening.

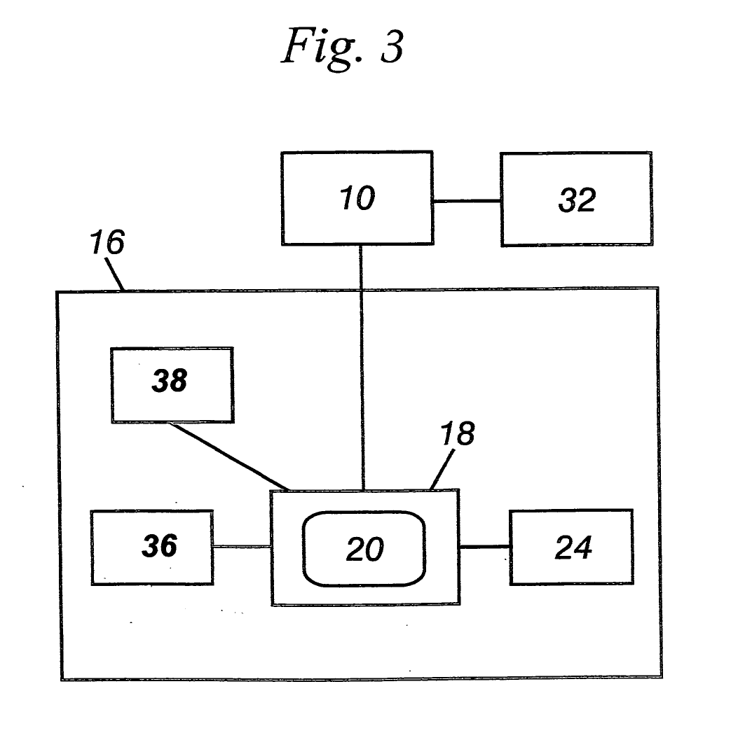

Balance screening will be integrated into an AuDChek device in a kiosk 16 as shown in FIG. 3 to assess the risk of balance problems and falls. The following screening tools are samples of balance screenings to be used, as well as balance screening using visual nystagmography goggles 36, connected to the computer. A questionnaire 38 about a person's balance, such as Activities Specific Balance Confidence Scale (ABC Scale) which is a quick questionnaire to determine balance confidence concerns/fear of falling, will be answered by the person with the touch screen or voice recognition of the AudChek device. Other tests will be performed during the screening session and the results will be inputted into AuDChek for referral and appointment scheduling online, for a complete balance assessment if the patient fails the screening. This is done in the same manner as for a hearing screening, as set forth above. Such other tests include, but are not limited to:
A. Timed Up & Go: a moderated movement test that is evaluated by an individual
B. Modified Clinical Test of Sensory Integration and Balance (CTSIB): the test is performed without the use of a dome.
C. Vibratory sensation screening: a 40 dB, 250 Hz tone is applied to the large toe and ankle bone of each lower extremity, and the patient is asked whether the vibration is felt. A base line is obtained on the large thumb first—so that the person can feel the vibration. The suspicion is high for neuropathy if the individual is unable to feel the vibration when applied to the lower extremities.
D. Bedside vestibular examination—head thrusts: The patient stares at the examiner's nose while the patient's head is quickly rotated in an arc (15° peak movement) to the right or left side. Any indications of a corrective saccade after movement of the head indicates reduced vestibulo-ocular reflex gain.

A head shake balance screening test can be performed with the eye chart of the vision screening (see below) to assess occilopsia, a vestibular problem brought on by vestibulotoxicity. The results will be inputted into AuDChek along with patient information, which are merged, then transmitted to a doctor or medical clinic for appointment scheduling for complete balance assessment, upon failure of the test by the patient.

Vision screening will be performed using best correction (glasses or contacts). Screening at the test site of the AuDChek screening with an eye chart 40 posted in the room of the screening device will indicate pass/fail results (greater than 20/40 vision), preferably an automated eye chart connected to the computer 18. Responses will be entered into AuDChek during the vision screening either by touch screen 20 or voice recognition. In addition, vision screening may be accomplished by connecting an Stereo Optics Optec 5000 Vision Tester 42 to the AuDChek device. The Optec is a simple test sequence that can be administered by ancillary office staff in less than 5 minutes. Standard test packages are designed for use in multiple areas, including occupational medicine, school, athletics, F.A.A. (Federal Aviation Administration), drivers licensing, public health, primary care, D.O.T. (Department of Transportation), driver rehabilitation, and contrast sensitivity.

Failures on the eye chart or Optec device will be indicated in AuDChek and a referral and appointment scheduled with an optometrist, ophthalmologist or eye clinic for a complete eye evaluation in the same manner as in the hearing screening procedure.

### SUMMARY OF THE ACHIEVEMENT OF THE OBJECTS OF THE INVENTION

From the foregoing, it is readily apparent that I have invented an improved method and apparatus for determining the hearing capability of a person, which provides and conducts an automated hearing test and determines the responses to the test; an apparatus that screens the hearing capability of a person; that incorporates a touch screen for response to hearing screening stimuli; that prints out results with a doctor's signature line, as needed at this point for insurance coverage of future diagnostic hearing testing; that is linked online to a specific nearby clinic schedule where appointments can be made immediately upon failing a hearing screening; that incorporates a web portal with an Internet based scheduler for the local contracted clinic where, immediately on failing the hearing screening, the patient schedules a convenient, open time to have a full hearing evaluation; and that provides printed reports, online scheduling and automated testing in a single apparatus for screening hearing.

It is to be understood that the foregoing description and specific embodiments are merely illustrative of the best mode of the invention and the principles thereof, and that various modifications and additions may be made to the apparatus by those skilled in the art, without departing from the spirit and scope of this invention, which is therefore understood to be limited only by the scope of the appended claims.

What is claimed is:

1. A method of determining the hearing capability of a person, comprising:
providing and conducting an automated hearing test of such person utilizing a computer;
determining the responses to the test;
determining and recording the results of the test as pass or fail;
obtaining personal information from such person;
merging and storing the test results and the personal information in the computer; and
transmitting such merged results and information to an audiologist, health care provider, medical office, or clinic.

2. A method according to claim 1, further comprising providing a computerized questionnaire concerning hearing health to the person, and recording the person's responses in a computer.

3. A method according to claim 1 wherein the automated hearing test is selected from the group consisting of: an automated air conduction pure tone hearing test, a narrow band hearing test, a speech in noise test, or a speech word test.

4. A method according to claim 1, further comprising providing an online scheduling portal for a scheduling hearing appointment with an audiologist, medical office, or clinic.

5. A method according to claim 1, further comprising providing both verbal and visual instructions by computer to the person being tested.

6. Apparatus for determining the hearing capability of a person, comprising:
a computer:
automated hearing test software loaded into said computer;
means for determining responses to the test and associated results;
means for collecting personal information;
means for indicating the results of the test as pass or fail;
means for merging and storing test results and personal information; and
means for transmitting such results and information to an audiologist, health care provider, medical office, or clinic.

7. Apparatus according to claim 6, wherein said computer is provided with a touch screen for the person to input information into the computer.

8. Apparatus according to claim 6, further comprising associated noise-canceling headphones connected to said computer.

9. Apparatus according to claim 8, wherein said noise-canceling headphones are circumaural headphones.

10. Apparatus according to claim 6, further comprising means associated with said computer for scheduling an appointment.

11. Apparatus according to claim 6, wherein said means for transmitting information is linked to an online scheduling portal.

12. Apparatus according to claim 11, wherein said means for transmitting information is a secure internet connection according to NIST SP 800-53 and HIPAA regulations.

13. Apparatus according to claim 6 wherein said computer has an associated printer.

14. Apparatus according to claim 6 further comprising a housing for said apparatus.

15. Apparatus according to claim 14, where said housing is a kiosk.

16. Apparatus for determining the vision capability of a person, comprising:
a computer and associated eye chart:
automated vision test software loaded into said computer;
means for determining responses to the test and associated results;
means for collecting personal information;
means for indicating the results of the test as pass or fail;
means for merging and storing test results and personal information; and
means for transmitting such results and information to an optometrist or ophthalmologist, or eye clinic.

17. Apparatus for determining the balance stability of a person, comprising:
a computer:
automated balance test software loaded into said computer;
means for determining responses to the test and associated results;
means for collecting personal information;
means for indicating the results of the test as pass or fail;
means for merging and storing test results and personal information; and
means for transmitting such results and information to a doctor, health care provider, or medical clinic.

18. Apparatus for determining the balance stability of a person according to claim 17, further comprising visual nystagmography goggles connected to the computer,
